(19) United States
(12) Patent Application Publication
(10) Pub. No.: US 2007/0055256 A1
(43) Pub. Date: Mar. 8, 2007

(54) Intraosseous fixation device for reconstructing the knee posterior cruciate ligament

(76) Inventor: Sergio Luiz Galindo, Sao Paulo (BR)

Correspondence Address: STITES & HARBISON PLLC, 1199 NORTH FAIRFAX STREET, SUITE 900, ALEXANDRIA, VA, 22314, US

(21) Appl. No.: 11/412,128

(22) Filed: Apr. 27, 2006

(30) Foreign Application Priority Data
Aug. 22, 2005 (BR) P10503558-5

(51) Int. Cl. A61B 17/58

(52) U.S. Cl. 606/72

(57) ABSTRACT

An intraosseous fixation device for reconstructing the knee posterior cruciate ligament, comprising a monoblock body (1), preferably made of titanium, formed by a rod (2) that, by one end, is widened to form a tab (3) being a point to receive the double semitendinosus and gracilis cross graft, while, in the opposite side, said rod is integrated to an eyelet (4) having a cooperating geometry to receive the low profile cortical screw and cooperating so that this end is embedded in the tibia.

### BACKGROUND OF THE INVENTION

1. Field of the Invention

The present invention relates to a new surgical component constituting new means for tibial rigid fixation for the knee posterior cruciate ligament reconstruction grafts.

2. Description of the Prior Art

As it is known, the main and most complex joint of the human body is undoubtedly the knee joint, since it combines three different bones: the femur, the tibia and the patella. These three bone structures form two joints: the femoropatellar joint and the tibiofemoral joint that functionally cannot be always considered in separate, since there is a mechanical relationship between them. The delimiter of this joint consists in the femur distal end, the tibia proximal end with interposed menisci imparting the joint symmetry. The joint is surrounded by muscles, ligaments, menisci and joint capsule acting together to harmonize the flexion and extension movements with low rotation. Its function is related to its integrated muscular activity and its restrictive and precise ligamentous structures.

The knee joint contains the two cruciate ligaments, that is, anterior and posterior, both normally simply indicated as ACL and PCL. They are called cruciate since they cross each other in the center of the knee joint, where both are combined to prevent the tibia anterior or posterior translation movement in relation to the femur (sagittal instability control).

It is notorious that the knee diseases may have different anatomical seats and reach several age ranges, being also caused by several different factors, such as sport traumas, home accidents, organic diseases and degenerative diseases.

Among several diseases, one of them has a particular relationship with the present invention, that is, those generically diagnosed as “sagittal instability control problems”, caused by posterior cruciate ligament injuries. Such injuries, in some cases, are treated with physiotherapy, however, in other cases, a surgical procedure is inevitable, especially in case of ligament rupture.

The knee anterior cruciate ligament (PCL) [SIC] rupture is related to different activities, particularly sports, and may be also the result of different kinds of occupational or home accidents.

On the other hand, the PCL ligament rupture is a frequent, incapacitating and progressing injury. The surgical treatment has been a highly reliable recovery process for more than 15 years and physiotherapy has an essential role in complementing it.

Some current surgical techniques widely used for reconstructing the cruciate ligament are applied using arthroscopy. Most of the time, it is possible to use an autologous graft (a tendon from the patient him/herself): the most used grafts are the patellar tendon medial ⅓, goose foot tendons or knee flexors (semitendinosus and gracilis) or the fascia lata tendon. This tendon is positioned in the place of the ruptured anterior cruciate ligament (anatomical position). The chosen transplant is fixated to the femur and the tibia using interference screws or other similar fixation systems.

Therefore, in the present art, the grafts are fixated to the tibial and femoral tunnels using components in the form of interference screws.

Since 1919 when Hey Groves proposed the surgical ligament reconstruction by using the iliotibial tract performing an intra- and extra-articular repair and fixating the graft with two screws at the tibia level (The Knee Lewin 1952, page 301), the ACL injury treatment evolution has been subject to a number of changes as a result of the increased knowledge of the biophysical features of the used structures.

An important point in the ACL or PCL ligament reconstruction study was the better knowledge of the biological structures features chosen to replace the injured ligaments.

It is known today that the biological structures as the semitendinosus and gracilis tendon, the patellar tendon or the quadricipital tendon (those more often studied) have biophysical features that are similar to the anterior ACL and posterior PCL cruciate ligaments.

After choosing the graft for replacing the injured ACL or PCL, the great difference consists in choosing a fixation method. This fixation method shall have a strength that is close to the original fixation strength of said structures.

The rigid fixation of the implanted ligament is regarded as one of the most important factors for a successful ligament reconstruction, mainly when using the knee flexor tendons.

The tibial fixation is considered more problematic than the femoral fixation, mainly because of the lower quality of the tibia metaphyseal bone when compared to the femur and due to the difficulty in fixating the four DSTG bundles.

Upon the fixation to the tibia, other factors are also important: the length of the tendon inside the bone tunnel, the graft distance from the joint and the bone consistency where the tunnel is located.

Undoubtedly, the ligament reconstruction surgical procedures show appropriate efficiency levels. However, as previously mentioned, the tibial fixation is considered as the most troublesome and, as a consequence, the components currently used shall have qualities that offset the lower quality of the tibia metaphyseal bone. Otherwise, the surgical success may be impaired by the fixation component.

Due to the high incidence of ACL injuries, 100,000 every year in the United States, 5,500 in Australia having a population of 20,000,000, surgical treatments for this kind of injury became popular. Techniques using several grafts and affixation means have been improved.

In the femur, the cross fixations, Bone Mulch (1112 N), Rigid Fix (868 N), Endobutton CL (1086 N), BioSrew (589 N), have showed excellent results in the treatment of ACL injury.

Since the tibia bone has lower metaphyseal quality and density than the femur and is difficult to fixate four bundles of the DSTG, the fixation means show much lower results mechanically than at the femur level.

In 2003, Petter Kousa et al presented a randomized work with a comparative study of six fixation means and concluded:

The fixation using Intrafix (1332 N) had a better performance than those using WasherLoc (975 N), Tandem spiked washer (769 N), SmartScrew (665 N), Bioscrew (612 N) and SoftSilk (471 N).

### SUMMARY OF THE INVENTION

The main object of the invention is to provide an intraosseous fixation device having completely different technical features compared to those currently used. Said differences cover the physical aspects of the piece itself, and also characterizes a new fixation solution, since the device has a completely different constructive aspect, that is, it is a straight rod having an end with an oblong eyelet shaped as a link, while in the opposite end another eyelet is angularly positioned that, in addition to be circular and tilted, also has a shape to receive a cortical screw, so that this end may be fixated to the tibia, while the other one is directed upwards, configuring means for the graft transposing.

Therefore, with the device of the present invention, a new fixation method is defined as characterized for being an intra-tunnel cross fixation; having the titanium stiffness and elasticity; having a varying length and for being fixated to the tibia with a cortical screw.

The present seeks to provide an intraosseous fixation device for reconstructing the knee posterior cruciate ligament, comprising a monoblock body (1), having a first end forming a tab (3) with two side portions, and constructed and arranged to receive a double semitendinosus and gracilis cross graft; a second end defining an eyelet (4) having a lower face and an upper face, constructed and arranged to receive a low profile cortical screw and to be affixed to a tibia, and a rod (2), connecting both ends.

As previously mentioned, the present fixation device is made of titanium and consists in a monoblock-type piece with a chain link-type oval-shaped tab having a diameter preferably ranging from 7 mm to 9 mm of 7, 8 or 9 mm, a rod also with a length preferably ranging from 40 mm to 50 mm, and a round tab for the low profile cortical screw fixation to the tibia. The oval-shaped tab principle is to receive the double semitendinosus and gracilis cross graft that may replace the anterolateral and posteromedial bundles or further a single tunnel in the femur that should reconstruct the anterolateral bundle. The double semitendinosus and gracilis grafts are passed through the oval-shaped hole measuring from 7 to 9 mm depending on the graft diameter that will be positioned inside the previously made and measured tunnel. For this tunnel with measured length, it should be placed by a fixation device that always allows a minimum graft length of 3.5 cm, that is, in case of a 7.5 cm tunnel, the present fixation device shall have a length of 4.0 cm, thus allowing 3.5 cm of graft. After passing the STG tendons in the femoral tunnels, the fixation device is fixated in the tibia using a bi-cortical screw. After this fixation, the fixation to the femur is performed.

The present fixation device defines a fixation method developed so as to use the double semitendinosus and gracilis tendons transversely and intra-tunnel.

This is a rigid structure made of titanium and having preferably three different lengths, being highly important since it can keep a constant relative to the graft length inside the bone tunnel, thus keeping a contact area between the tendinous graft and the bone surface, thus facilitating its integration. Another advantage is that, if the graft is short, the surgeon may use a longer IKDP, thus avoiding the grafts to be short and do not fill the femoral tunnels.

Finally, the fixation device is fixated to the tibia using the low profile cortical screw that should fixate two corticals and will avoid a salience at the skin level.

### BRIEF DESCRIPTION OF THE DRAWINGS

For a better understanding, the present invention is described in detail below, with reference to the attached drawings, wherein:

FIGS. 1, 2 and 3 show perspectives in different angles showing the constructive particulars of the present fixation device; and

FIGS. 4a to 12c show illustrations of the surgical technique used with the present fixation device.

### DETAILED DESCRIPTION OF THE INVENTION

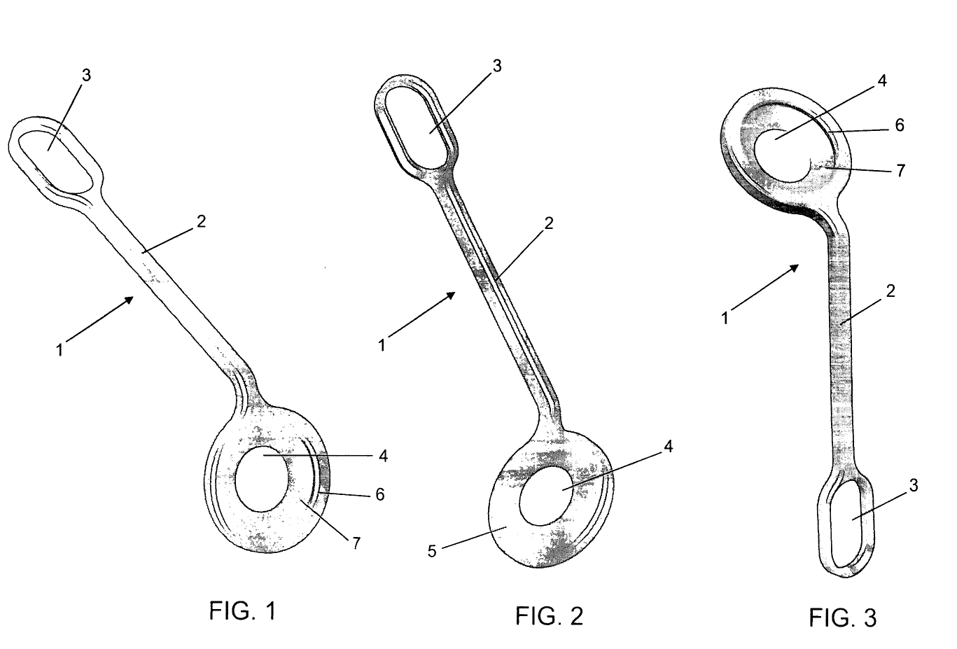

According to these drawings and their details, more particularly FIGS. 1, 2 and 3, the present invention, the intraosseous fixation device for reconstructing the knee posterior cruciate ligament of this invention is characterized in that it comprises a monoblock body (1), made of titanium, formed by a rod (2) that, by one end, is widened to form a tab (3) being a point to receive the double semitendinosus and gracilis cross graft, while, in the opposite side, said rod is integrated to an eyelet (4) having a cooperating geometry to receive the low profile cortical screw and cooperating so that this end is fixated to in the tibia.

The rod (2) shows a preferably quadrangular cross section with all corners being rounded and chamfered, this also occurring with the tab (3) and the eyelet (4).

The size of rod (2) ranges from 40 to 50 mm in length independently of its thickness and height.

The tab (3) is coplanar relative to rod (2), also having a preferably elongated shape lengthwise the rod (2), also having appropriate rounded ends matching their side portions.

The diameter of the tab (3) preferably ranges from 7 to 9 mm.

The eyelet (4) is angularly positioned relative to rod (2), its bottom face (5) being flat, what does not occur with the top face, where there is a lowering (6) and a reamed hole-like portion (7), cooperating to house and embed the low profile cortical screw head in the tibia.

Surgical technique.

Examination under anesthesia—all ligaments and degrees of instability should be reexamined with the patient anesthetized. This is necessary to define the PCL insufficiency degree, if there are associated injuries, as well as a good documentation.

Arthroscopic inspection—the patient is positioned in dorsal decubitus, under local or general anesthesia. The pneumatic tourniquet is placed at the thigh root level and with pressure of 320 mmHg. Asepsis and antisepsis and placement of sterile surgical fields, with the knee being flexed at 90 degrees. Three ports are made for the arthroscopic inspection. We routinely use the lateral suprapatellar port for infusing the saline solute solution and the lateral port for optical placement. The third port is medial to the patellar tendon. If present, intra-articular injuries should be treated.

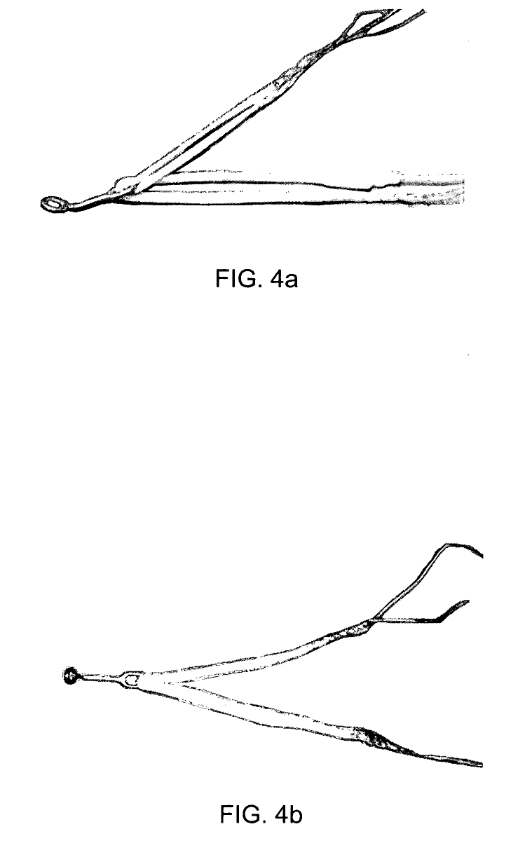

Grafts removal—an incision of approximately 4 to 6 cm is made at the goose foot insertion level. The semitendinosus and gracilis tendons (that are removed) are isolated. The semitendinosus and gracilis double tendons (FIGS. 4a and 4b) that are passed through tab (3) of the fixation device are prepared. The semitendinosus and gracilis tendons are independently sutured using resistant non-absorbable threads with a different color to facilitate identification.

The intra-tunnel cross fixation defined by the present fixation device has a length varying from 4.0 cm to 5.0 cm, increasing with 0.5 cm intervals and the oval-shaped ring where the tendons pass, has a diameter ranging from 7 mm to 9 mm. After fixating the grafts in the device, the grafts are pre-tensioned.

PCL stump debridement—the arthroscope is placed through the lateral port, the PCL debris is removed. At the femur level, we allow a minimum stump to direct the guide placement. In the routine tibia, we use an accessory port (posteromedial) for better viewing the tibia posterior region. This positioning facilitates the stump removal from the PCL of existing cicatricial fibrosis.

Positioning of the guide wires and tunnels perforation—the tibial guide uses the grafts removal incision in the tibia anteromedial region, is positioned at an angle ranging from 65 to 55 degrees. The guide wire size is delimited and positioned (should remain 1.0 or 1.2 cm from the tibia joint surface level).

When directly viewed, a spatula is used for protecting the vasculonervous structures of a surgical accident.

Next, the femur guide wires are positioned

The anterolateral guide wire is the first to be positioned inward, the knee should be flexed 90 degrees. The wire should be positioned in the top and anterior face of the femoral insertion point, approximately 12 to 10 mm from the posterior end of the joint cartilage of the medial femoral condyle when viewed from inside the knee. Comparing to a clock, the guide wire would be in right knee at 13:30 o'clock and in the left knee at 10:30 o'clock.

The extra-articular position of the guide is placed approximately 8 mm or 10 mm behind the joint cartilage of the medial femoral condyle.

Next, the posteromedial bundle guide wire are positioned, located 5 mm posterior and 5 mm distal relative to the anterior tunnel. Again, the extra-articular guide positioning should be 5 mm more inferior and posterior than the anterolateral bundle.

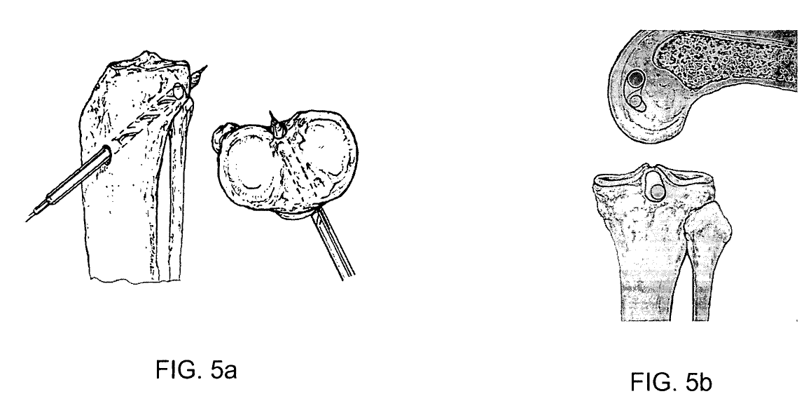

Next, FIGS. 5a and 5b, a tibial tunnel is made using a drill having the same diameter of the grafts. All of these are performed under direct viewing through the patellar trans-tendon or posteromedial port. Always remember to use precise instruments to protect the guide wire and the drill exit due to the presence of noble structures of the popliteal region. It is recommend the use of a fluoroscope (if available) during the surgery. With the tunnel perforated, its length is measured that should oscillate from 6.5 cm to 8 cm. As the grafts length is also known, an intra-tunnel fixation defined by the fixation device to allow the graft to remain inside the tunnel according to a minimum length of 3.5 cm. If the graft is short, the intra-tunnel fixation defined by a longer fixation device is used.

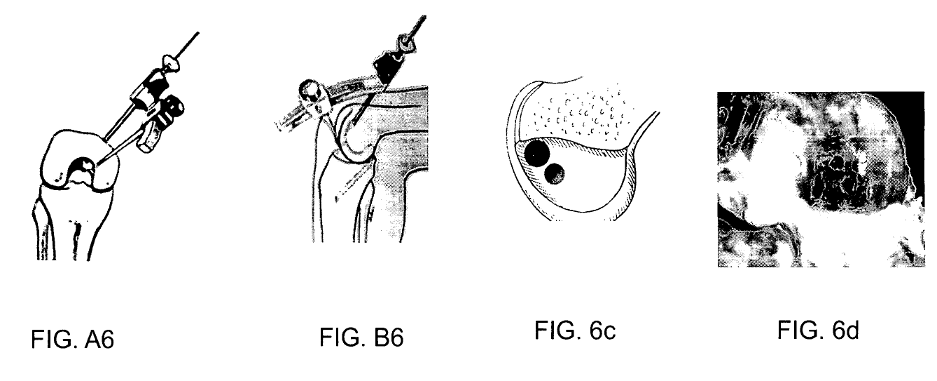

Next, as schematically depicted in FIGS. 6a to 6d, the femoral tunnels are perforated having a diameter according to the grafts diameter. The anterolateral bundle tunnel always ranges from 8 mm to 9 mm, while the posteromedial tunnel ranges from 6 mm to 7 mm.

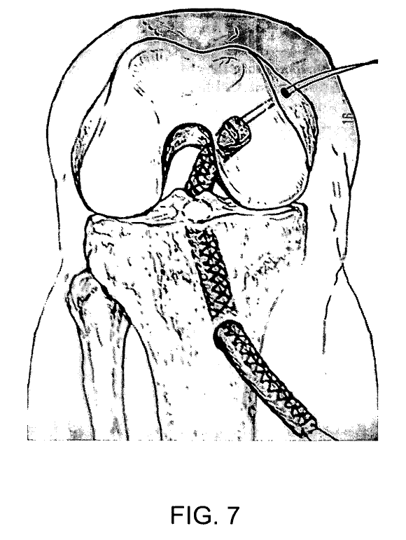

Grafts passage—when tunnels are made, the tibial tunnel (FIG. 7) is dilated with the Gore-smooth that, when entering the tibia region, should be brought with the aid of a grasper-type forceps through the port to the medial patellar. The tunnel is dilated by removing all irregularities. The non-absorbable fiberwire or maxbraid suture threads or the like (having different colors), are fixed to the Goresmooth and brought by the medial parapatellar port. With the aid of a duly identified grasper, the semitendinosus graft is passed through the anterolateral tunnel in the femur and the gracilis graft is passed through the posteromedial tunnel.

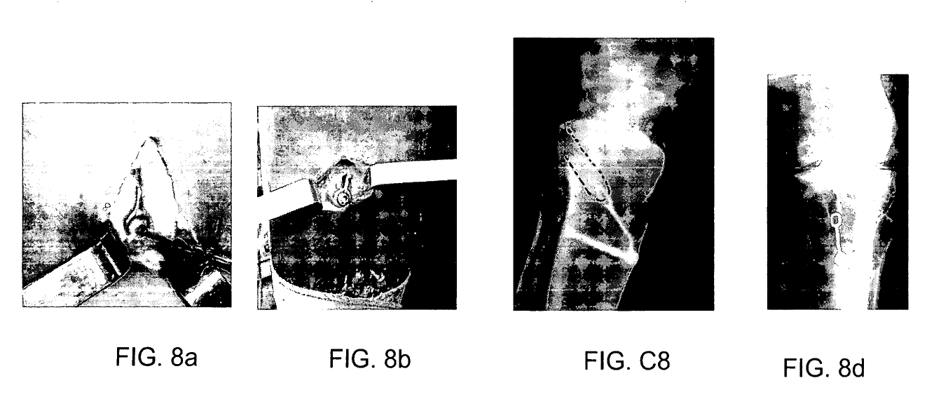

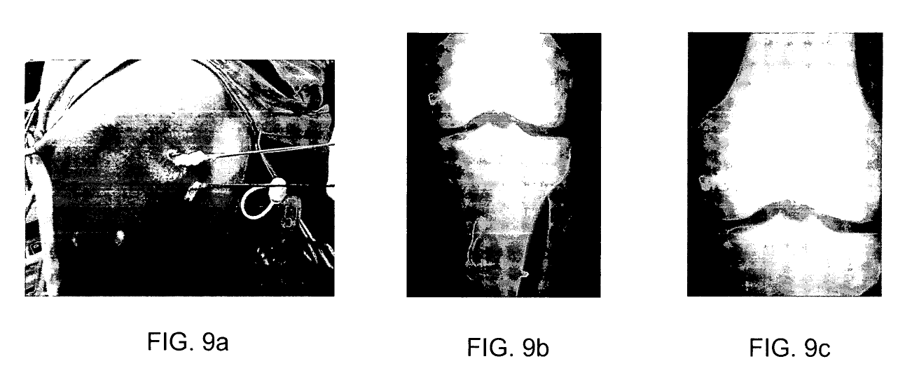

Grafts fixation—as showed in FIGS. 8a to 9c, once the femoral tunnel length and the grafts length are known, the device concerned is fixated through its eyelet (4) to the tibia using a low profile cortical screw. Then, the semitendinosus graft is pulled through the anterolateral tunnel, the tibia is anteriorized and the knee (at 90 degrees) is fixated using an interference screw for soft tissues.

With the knee flexed at 30 degrees and the tibia kept anteriorized, the gracilis graft is pulled through the posteromedial tunnel and is fixated using another interference screw.

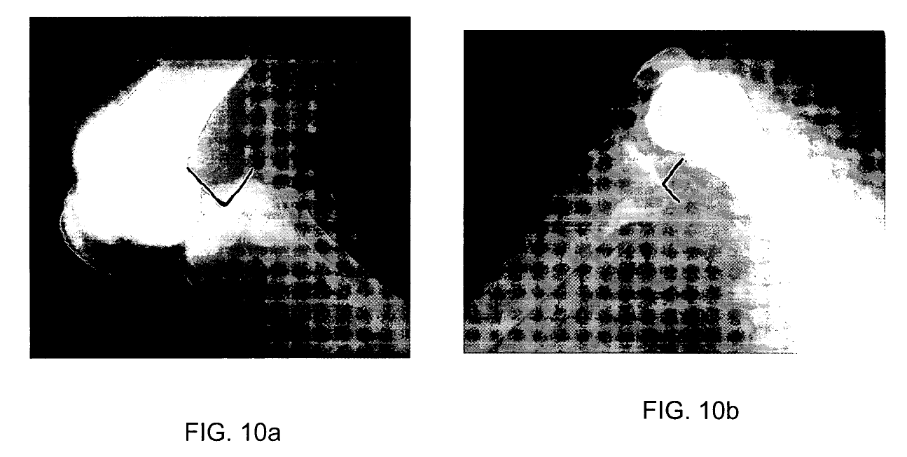

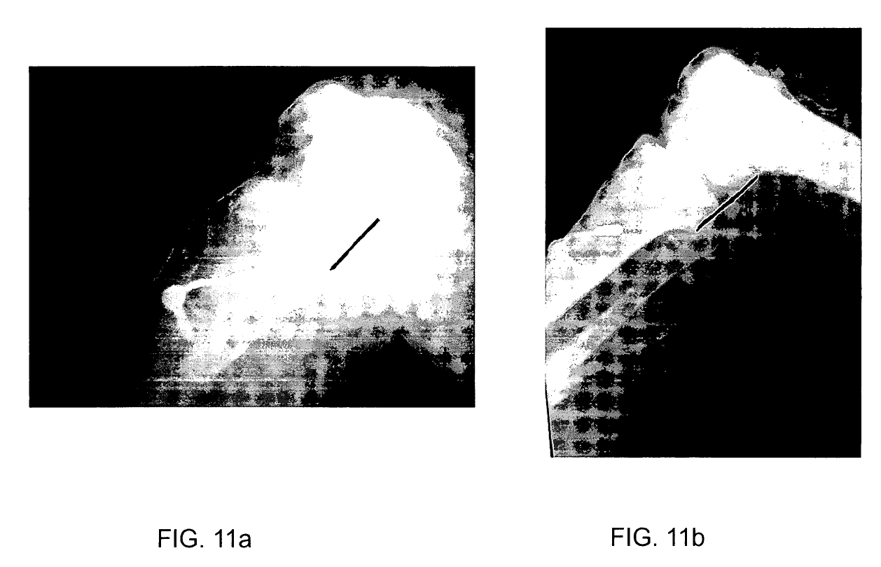

FIGS. 10a-10b and 11a-11b respectively show the pre- and postoperative conditions.

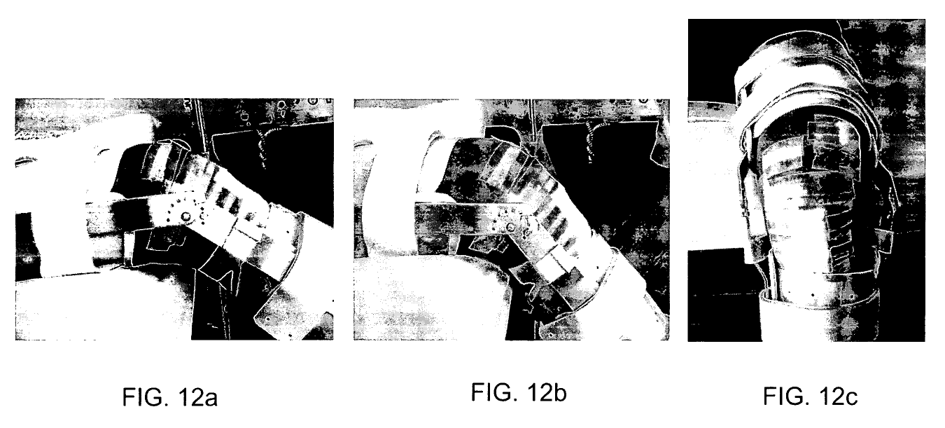

Postoperative and rehabilitation—as showed in FIGS. 12a to 12c, the patient remains with a knee immobilizer that allow flexion and extension movements since he/she can stand the pain. This immobilizer is intended to avoid the tibia posteriorization during the grafts ligamenting. The tibial plateau has an inclination of approximately 8 degrees towards the posterior, there are six posterior muscle groups and the gravity force favors the tibia posteriorization. Load is allowed with the aid of crutches from hospital discharge usually after 48 hours and removal after two weeks.

Throughout the six-month rehabilitation program, all exercises are assisted, preferably in ventral decubitus.

1. An intraosseous fixation device for reconstructing the knee posterior cruciate ligament, comprising:
a monoblock body (1), having:
a first end forming a tab (3) with two side portions, and constructed and arranged to receive a double semitendinosus and gracilis cross graft;
a second end defining an eyelet (4) having a lower face and an upper face, constructed and arranged to receive a low profile cortical screw and to be affixed to a tibia, and
a rod (2), connecting both ends.

2. The intraosseous device according to claim 1, wherein the rod (2) has a quadrangular cross section with all corners being rounded and chamfered.

3. The intraosseous device according to claim 1, wherein the tab (3) has a quadrangular cross section with all corners being rounded and chamfered.

4. The intraosseous device according to claim 1, wherein the eyelet (4) has a quadrangular cross section with all corners being rounded and chamfered.

5. The intraosseous device according to claim 1, wherein the length of the rod (2) ranges from 40 to 50 mm, independently of its thickness and height.

6. The intraosseous device according to claim 1, wherein the tab (3) is coplanar in relation to the rod (2).

7. The intraosseous device according to claim 1, wherein the tab (3), has an elongated shape lengthwise in relation to the rod (2).

8. The intraosseous device according to claim 1, wherein the tab (3), has rounded ends matching the side portions

9. The intraosseous device according to claim 1, wherein the diameter of the tab (3) ranges from 7 to 9 mm.

10. The intraosseous device according to claim 1, wherein the eyelet (4) is angularly positioned in relation to the rod (2).

11. The intraosseous device according to claim 1, wherein the lower face (5) of the eyelet (4) is flat.

12. The intraosseous device according to claim 1, wherein the upper face (5) of the eyelet (4) includes a lowering (6) and a reamed hole-like portion (7), constructed and arranged to house and embed a low profile bicortical screw head in the tibia.

13. The intraosseous device according to claim 1, wherein the monoblock body (1) is made of titanium.
